SEQUENCE LISTING (7 sequences)

SEQ ID NO: 1 - protein, 15 aa, synthetic construct
LPPLLTDEMIAQYTS

SEQ ID NO: 2 - protein, 9 aa, synthetic construct
LTDEMIAQY

SEQ ID NO: 3 - protein, 9 aa, synthetic construct
YLQPRTFLL

SEQ ID NO: 4 - protein, 9 aa, synthetic construct
QYIKWPWYI

SEQ ID NO: 5 - protein, 9 aa, synthetic construct
RLQSLQTYV

SEQ ID NO: 6 - protein, 9 aa, synthetic construct
KCYGVSPTK

SEQ ID NO: 7 - protein, 8 aa, synthetic construct
SIINFEKL

(19) United States
(12) Patent Application Publication
(10) Pub. No.: US 2026/0069676 A1
(43) Pub. Date: Mar. 12, 2026

(54) PEPTIDE-LOADED ANTIGEN PRESENTING CELL-DERIVED EXTRACELLULAR BLEBS AS A MOLECULARLY TARGETED VACCINE

(71) Applicant: The Regents of the University of California, Oakland, CA (US)

(72) Inventors: Young Jik Kwon, Irvine, CA (US); Jee Young Chung, Irvine, CA (US)

(21) Appl. No.: 19/106,614

(22) PCT Filed: Aug. 31, 2023
(86) PCT No.: PCT/US2023/073255

§ 371 Date: Feb. 25, 2025

Related U.S. Application Data
(60) Provisional application No. 63/402,939, filed on Aug. 31, 2022.

(51) Int. Cl. A61K 39/215; A61K 39/00; A61P 31/14

(52) U.S. Cl. CPC A61K 39/215; A61P 31/14; A61K 2039/55505; A61K 2039/55555; A61K 2039/55561; C12N 2770/20034

(57) ABSTRACT

The disclosure provides for vaccine preparations comprising isolated or purified extracellular blebs that display engineered MHC I and MHC II peptides that target specific antigen(s) or a specific epitope(s) from a pathogen, and uses thereof, including for vaccination against the pathogen and disease.

### CROSS REFERENCE TO RELATED APPLICATIONS

This application claims priority under 35 U.S.C. § 119 from Provisional Application Ser. No. 63/402,939 filed Aug. 31, 2022, the disclosure of which is incorporated herein by reference.

### TECHNICAL FIELD

The disclosure provides for vaccine preparations comprising isolated or purified extracellular blebs that display engineered MHC I and MHC II peptides that target specific antigen(s) or a specific epitope(s) from a pathogen or a disease, and uses thereof, including for vaccination against the pathogen.

### BACKGROUND

Vaccines currently used against a disease generally consumes a lot of time for their production owing to their complicated and rigorous norms. For example, traditionally designed vaccines for first strain of COVID-19 have lost their efficacy as highly mutated SARS-CoV-2 strains have developed. To redesign these vaccines is time-consuming, thus the demand for effective vaccines would outstrip supply to an uncontrollable extent. As such, feasible alternatives to traditionally designed vaccines are needed.

### SUMMARY

The present disclosure relates to methods of preparing molecularly engineered extracellular blebs derived from antigen presenting cells such as dendritic cells, for use in various preventive and therapeutic treatments against infectious diseases and more. Specifically, the present disclosure relates to extracellular blebs obtained from peptide loaded bone marrow derived dendritic cells for enhanced, molecularly directed immunity mediated by both CD4 and CD8 T cell activation in a quantitatively orchestrated manner. More specifically, the compositions and methods presented herein optimize the presentation likelihood of a set of vaccine peptides to maximize vaccine immunogenicity against specific antigens or specific epitopes thereof. Additionally, the compositions and methods presented herein can also be used to investigate the roles of humoral vs. cellular immune response in disease prevention and therapy. In the studies presented herein, extracellular blebs obtained from dendritic cells that were molecularly engineered to present MHC class I and MHC II class molecules that were specific to peptides derived from the SARS-CoV-2 spike protein, promoted significant immunity against SARS-CoV-2 and its variants when administered in vivo. The methods and techniques disclosed herein to generate vaccines against SARS-CoV-2 can similarly be applied to generate vaccines or therapies against infectious pathogens (e.g., influenza) and noninfectious diseases (e.g., cancer).

In furtherance of the foregoing, the disclosure provides methods for preparing molecularly engineered extracellular blebs, the molecularly engineered extracellular blebs made therefrom, and the use of the molecularly engineered extracellular blebs in various preventive and therapeutic treatment against infectious diseases and more. In a further embodiment, the molecularly engineered material comprises extracellular blebs obtained from peptide loaded antigen presenting cells (e.g., dendritic cells) for enhanced immunity mediated by both CD4 and CD8 T cell activation in a quantitatively orchestrated manner. The methods of the disclosure optimize the presentation of a set of vaccine peptides to maximize vaccine immunogenicity and molecular specificity. Additionally, the method of the disclosure can probe the roles of humoral vs. cellular immune response in disease prevention and therapy. In the studies presented herein, SARS-CoV-2 spike protein-derived peptides binding to the MHC class I and MHC II class molecules of dendritic cells were used as model peptides that are capable of generating immunity against SAARS-CoV-2 and its variants. The same principle can be applied to other infectious pathogens (e.g., influenza) and noninfectious disease (e.g., cancer) prevention and therapy.

In some embodiments, the disclosure provides for a vaccine preparation comprising: isolated or purified extracellular blebs (EBs) that present engineered MHC I and MHC II peptides that target specific antigen(s) or a specific epitope(s) from a pathogen; wherein the EBs are isolated or purified from an antigen presenting cell. In another embodiment, the antigen presenting cell is selected from a dendritic cell, a macrophage, and a B-Cell. In yet another embodiment, the antigen presenting cell is a dendritic cell. In a further embodiment, the antigen presenting cell presents the engineered MHC I and MHC II peptides that target specific antigen(s) or a specific epitope(s). In yet a further embodiment, the pathogen is selected from a fungus, a virus, or a bacterium. In a certain embodiment, the pathogen is a bacterium selected from the following: Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis. In another embodiment, the pathogen is a fungus selected from the following: Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosurn, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii. In yet another embodiment, the pathogen is a virus selected from the following: Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus. KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastern chimpanzee simian foamy virus, Simian foamy virus, Dhori virus, Vientovirus, Human torovirus, Varicella-zoster virus, Chandipura virus, Isfahan virus, and Vesicular stomatitis virus. In a further embodiment, the Human SARS coronavirus is SARS-CoV-2, and/or a variant thereof. In yet a further embodiment, the specific antigen(s) comprises a peptide sequence for a portion of the spike protein from SARS-CoV-2 and/or a variant thereof. In another embodiment, the engineered MHC I peptide comprises the sequence of SEQ ID NO:2, SEQ ID NO:3, SEQ ID NO:4, SEQ ID NO:5 or SEQ ID NO:6. In yet another embodiment, the engineered MHC I peptide comprises the sequence of SEQ ID NO:2. In a further embodiment, the engineered MHC I peptide has a sequence that consists essentially of SEQ ID NO:2. In yet a further embodiment, the engineered MHC II peptide comprises the sequence of SEQ ID NO: 1. In another embodiment, the engineered MHC II peptide has a sequence that consists essentially of SEQ ID NO:1. In yet another embodiment, the vaccine preparation further comprises an adjuvant. In a further embodiment, the adjuvant is selected from aluminum hydroxide, aluminum phosphate, aluminum potassium sulfate, AS04, MF59, AS01_{B}, and CpG 1018. In yet a further embodiment, the vaccine preparation is formulated for intramuscular delivery, subcutaneous delivery, intradermal, or intranasal delivery. In another embodiment, the vaccine preparation is administered as a single dose, or as a primary dose with one or more follow up dose(s). In yet another embodiment, the vaccine preparation is administered as a primary dose with one or more follow up dose(s), where there is at least 7 days between the administration of each dose.

In some embodiment, the disclosure also provides a method of making a vaccine preparation disclosed herein, the method comprising: treating an antigen presenting cell that presents engineered MHC I and MHC II peptide sequences that target specific antigen(s) or a specific epitope(s) from a pathogen with a blebbing agent; isolating EBs from the antigen presenting cell; preparing a vaccine preparation comprising the isolated EBs. In another embodiment, the blebbing agent comprises paraformaldehyde, N-ethylmaleimide, or photosensitizers. In yet another embodiment, method further comprises: engineering MHC I and MHC II peptide sequences that target specific antigen(s) or a specific epitope(s) from a pathogen using a computational model of peptide vaccines for eliciting cellular immunity based upon the prediction of peptide presentation by HLA molecules from patients that were infected by the pathogen; and presenting the engineered MHC I and MHC II peptide sequences into an antigen presenting cell. In a further embodiment, the pathogen is selected from a fungus, virus, or bacterium. In yet a further embodiment, the pathogen is a bacterium selected from the following: Actinomyces israeli, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis. In another embodiment, the pathogen is a fungus selected from the following: Absidia corymbifera, Absidia ramose, Achorion gallinae, Actinomadura spp., Ajellomyces dermatididis, Aleurisma brasiliensis, Allersheria boydli, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattia, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albican, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii. In yet another embodiment, the pathogen is a virus selected from the following: Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus, KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastem chimpanzee simian foamy virus, Simian foamy virus, Dhori virus, Vientovirus, Human torovirus, Varicella-zoster virus, Chandipura virus, Isfahan virus, and Vesicular stomatitis virus. In a certain embodiment, the Human SARS coronavirus is SARS-CoV-2, and/or a variant thereof. In a further embodiment, the engineered MHC I peptide comprises the sequence of SEQ ID NO:2, SEQ ID NO:3, SEQ ID NO:4, SEQ ID NO:5 or SEQ ID NO:6. In yet a further embodiment, the engineered MHC I peptide comprises the sequence of SEQ ID NO:2. In another embodiment, the engineered MHC I peptide has a sequence that consists essentially of SEQ ID NO:2. In yet another embodiment, the engineered MHC II peptide comprises the sequence of SEQ ID NO: 1. In a further embodiment, the engineered MHC II peptide has a sequence that consists essentially of SEQ ID NO:1.

In some embodiments, the disclosure further provides a method for vaccinating a subject against a pathogen, comprising: administering to the subject one or more doses of the vaccine preparation of the disclosure. In another embodiment, the pathogen is selected from a fungus, a virus, or a bacterium. In yet another embodiment, the pathogen is a bacterium selected from the following: Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis. In a further embodiment, the pathogen is a fungus selected from the following: Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporumfurfur, Pneumocystis jirovecii (or Pneumocystis carinit), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii. In a further embodiment, the pathogen is a virus selected from the following: Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus, KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastem chimpanzee simian foamy virus, Simian foamy virus, Dhori virus, Vientovirus, Human torovirus, Varicella-zoster virus, Chandipura virus, Isfahan virus, and Vesicular stomatitis virus. In yet a further embodiment, the Human SARS coronavirus is SARS-CoV-2, and/or a variant thereof. In another embodiment, the vaccine preparation comprises an adjuvant or is co-administered with an adjuvant. In yet another embodiment, the adjuvant is selected from aluminum hydroxide, aluminum phosphate, aluminum potassium sulfate, AS04, MF59, ASO1_{B}, and CpG 1018. In a further embodiment, the vaccine preparation is administered intramuscularly, subcutaneously, intradermally, or intranasally to the subject. In yet a further embodiment, the vaccine preparation is administered to the subject as a single dose, or as a primary dose with one or more follow up dose(s). In a particular embodiment, the vaccine preparation is administered to the subject as a primary dose with one or more follow up dose(s), where there is at least 7 days between the administration of each dose.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1A-C provides an in silico computational approach for MHC I and MHC II T cell epitopes. (A) Presents the best performing overlapping MHC-I and MHC-II peptide sequence, LPPLLTDEMIAQYTS (SEQ ID NO:1), against the epitope, LTDEMIAQY (SEQ ID NO: 2), from 5 peptide sequences that was tested using an in silico computational approach and in vitro experiments. (B-C) Results of testing of five overlapping MHC-I and MHC-II peptide sequences in silico and in vitro experiments. The five overlapping MHC-I and MHC-II peptide sequences being: LTDEMIAQY (SEQ ID NO: 2), YLQPRTFLL (SEQ ID NO: 3), QYIKWPWYI (SEQ ID NO:4), RLQSLQTYV (SEQ ID NO:5), and KCYGVSPTK (SEQ ID NO:6). As shown, the sequence presented in (A) was the best performer in the in vitro experiments.

FIG. 2 provides flow cytometry analysis of DC2.4 cells whose MHC I molecules were pre-loaded with SIINFEKL (SEQ ID NO:7), followed by replacing with LTDEMIAQY (SEQ ID NO:2) at varying concentrations. The optimized loading of the LTD peptide (MHC I) in DC 2.4 cells was found to be 1 μg/mL.

FIG. 3 shows pre-loaded SIINFEKL and SIINFEKL+LTDEMIAQY (SEQ ID NO:7 and SEQ ID NO:2) peptide on dendritic cells that were induced for blebbing using NEM and PFA. The isolated blebs were analyzed for flow cytometry using SIINFEKL (SEQ ID NO:7) antibody. LTD corresponds to LTDEMIAQY (SEQ ID NO:2).

FIG. 4A-D shows the immunization of mice by MHC I and MHC II peptide loaded EBs and antibody. (A) Vaccination of the C57/BL6 mice scheme. (B) Extracellular bleb production from dendritic cells. (C-D) Anti-spike antibody in the plasma from the vaccinated mice with PBS, free MHC I (1 μg/mL) and MHC II peptide (100 μg/mL), 2.5×10⁵ MHC I and MHC II peptide loaded BMDCs and MHC I and MHC II peptide loaded DC EBs at an equivalent surface area of 2.5×10⁵ of the parental cells at (C) day 14 and (D) day 24.

FIG. 5A-C presents the results of a D14 neutralization assay by MHC I and MHC II peptide loaded BMDCs and EBs. Mice were vaccinated as explained in FIG. 2C figure legend. The anti-sera were co-incubated with pseudotyped-SARS-CoV-2 spike (A) alpha, (B) delta and (C) omicron virus. The mean fluorescence intensity was determined by measuring the GFP expression.

FIG. 6A-C presents the results of a D24 neutralization assay by MHC I and MHC II peptide loaded BMDCs and EBs. Mice were vaccinated as explained in FIG. 4C figure legend. The anti-sera were co-incubated with pseudotyped-SARS-CoV-2 spike (A) alpha, (B) delta and (C) omicron virus. The mean fluorescence intensity was determined by measuring the GFP expression.

FIG. 7 shows specific lysis in splenocytes before peptide activation. Representative plots for specific lysis of EL4 spike cells by the splenocytes harvested from the mice 10 days after vaccination with PBS, free MHC I (1 μg/mL) and MHC II peptide (100 μg/mL), 2.5×10⁵ MHC I and MHC II peptide loaded BMDCs and MHC I and MHC II peptide loaded DC EBs at an equivalent surface area of 2.5×10⁵. EL4-spike cells were labeled with cell trace blue and incubated with splenocytes at 25:1 E:T ratio for 4 h and the specific lysis was determined by the percentage of YO-pro-1 positive cells by flow cytometry.

FIG. 8 demonstrates specific lysis in splenocytes after peptide activation. Representative plots for specific lysis of EL4 spike cells by the splenocytes harvested from the mice 10 days after vaccination with PBS, free MHC I (1 μg/mL) and MHC II peptide (100 μg/mL), 2.5×10⁵ MHC I and MHC II peptide loaded BMDCs and MHC I and MHC II peptide loaded DC EBs at an equivalent surface area of 2.5×10⁵. The splenocytes were activated overnight with MHC I peptide (10 μg/mL) and plated with EL4-spike cells at 25:1 E:T ratio for 4 h and the specific lysis was determined by the percentage of YO-pro-1 positive cells by flow cytometry.

### DETAILED DESCRIPTION

As used herein and in the appended claims, the singular forms “a,” “an,” and “the” include plural referents unless the context clearly dictates otherwise. Thus, for example, reference to “an extracellular bleb” includes a plurality of such blebs and reference to “the epitope” includes reference to one or more epitopes and equivalents thereof known to those skilled in the art, and so forth.

Also, the use of “or” means “and/or” unless stated otherwise. Similarly, “comprise,” “comprises,” “comprising”, “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

Other than in the operating examples, or where otherwise indicated, all numbers expressing quantities of ingredients or reaction conditions used herein should be understood as modified in all instances by the term “about.” The term “about” when used to describe the present invention, in connection with percentages means±1%.

The terms “blebbing”, “plasm membrane blebbing” or “cell membrane blebbing” as used herein, all refer to methods disclosed herein that induce plasma membrane blebbing in cells resulting in the production of extracellular blebs. Typically, blebbing of the plasma membrane is a morphological feature of cells undergoing late-stage apoptosis. A bleb is an irregular bulge in the plasma membrane of a cell caused by localized decoupling of the cytoskeleton from the plasma membrane. The bulge eventually separates from the parent plasma membrane taking part of the cytoplasm with it to form an extracellular bleb. Blebbing is also involved in some normal cell processes, including cell locomotion and cell division. Cell blebbing can be manipulated by mechanical or chemical treatment. It can be induced following microtubule disassembly, by inhibition of actin polymerization, increasing membrane rigidity or inactivating myosin motors, and by modulating intracellular pressure. Extracellular blebs can also be induced in response to various extracellular chemical and physical stimuli, such as exposure to agents that bind up sulfhydryl groups (i.e., sulfhydryl blocking agents).

The term “blebbing agent”, as used herein refers to chemical agents, such as sulfhydryl blocking agents, that when administered to cells induce the cells to undergo plasma membrane blebbing.

The term “sulfhydryl blocking agent” as used herein, refers to compound or reagent that interacts with cellular sulfhydryl groups so that the sulfhydryl group is blocked or bound up by the sulfhydryl blocking agent, typically via alkylation or disulfide exchange reactions. Chemical agents that can be used in the methods or compositions disclosed herein that block or bind up sulfhydryl groups includes, but are not limited to, mercury chloride, p-chloromercuribenzene sulfonic acid, auric chloride, p-chloromercuribenzoate, chlormerodrin, meralluride sodium, iodoacetamide, paraformaldehyde, dithiothreitol and N-ethylmaleimide.

The term “a sulthydryl blocking agent that results in extracellular bleb production” or the like as used herein, refers to a small molecule compound that when administered induces plasma membrane blebbing in cells, usually by causing injuries to cells by binding up or blocking sulfhydryl groups of biomolecules, such as proteins.

The term “molecularly engineered extracellular bleb” as used herein refers to an extracellular bleb that presents engineered peptide (e.g., MHC I and MHC II) sequences that target specific antigen(s). In some embodiments, a “molecularly engineered extracellular bleb” refers to an extracellular bleb that presents engineered MHC I and MHC II peptide sequences that targets a specific epitope of a targeted antigen.

The term “effective amount” as used herein, refers to an amount that is sufficient to produce at least a reproducibly detectable amount of the desired result or effect. An effective amount will vary with the specific conditions and circumstances. Such an amount can be determined by the skilled practitioner for a given situation.

The terms “patient”, “subject” and “individual” are used interchangeably herein, and refer to an animal, particularly a human, to whom treatment including prophylaxis treatment is provided. This includes human and non-human animals. The term “non-human animals” and “non-human mammals” are used interchangeably herein includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. A subject can be male or female. A subject can be a fully developed subject (e.g., an adult) or a subject undergoing the developmental process (e.g., a child, infant or fetus).

The term “therapeutically effective amount” as used herein, refers to an amount that is sufficient to affect a therapeutically significant reduction in one or more symptoms of the condition when administered to a typical subject who has the condition. A therapeutically significant reduction in a symptom is, e.g., about 10%, about 20%, about 30%, about 40%, about 50%, about 60%, about 70%, about 80%, about 90%, about 100%, or more as compared to a control or non-treated subject.

The term “treat” or “treatment” as used herein, refers to a therapeutic treatment wherein the object is to eliminate or lessen symptoms. Beneficial or desired clinical results include, but are not limited to, elimination of symptoms, alleviation of symptoms, diminishment of extent of condition, stabilized (i.e., not worsening) state of condition, delay or slowing of progression of the condition.

The terms “specific binding” or “specifically binding” when used in reference to the interaction of an antibody and a protein or peptide or an epitope and an MHC haplotype means that the interaction is dependent upon the presence of a particular structure (i.e., the antigenic determinant or epitope) on the protein; in other words, the antibody is recognizes and binds to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody. An example of epitope would include the portions of the spike protein from a virus (e.g., a coronavirus).

An “epitope” is the surface portion of an antigen capable of eliciting an immune response and of combining with the antibody produced to counter that response, or a T-cell receptor.

The term “isolated” when used in relation to extracellular blebs, as in “isolated extracellular blebs” refers to extracellular blebs that are separated from at least one contaminant with which it is ordinarily associated in its natural source, such as cells or cellular debris. Isolated extracellular blebs directly result from use of the blebbing agents taught herein and are therefore different from extracellular vesicles that are found in nature.

An “effective amount” is an amount sufficient to effect beneficial or desired results. An effective amount can be administered in one or more administrations,

The term “MHC subunit chain” as used herein refers to the alpha and beta subunits of MHC molecules. An MHC II molecule is made up of an alpha chain which is constant among each of the DR, DP, and DQ variants and a beta chain which varies by allele. The MHC I molecule is made up of a constant beta macroglobulin and a variable MHC A, B or C chain.

As used herein, the term “purified” or “to purify” refers to the removal of undesired components from a sample. As used herein, the term “substantially purified” refers to extracellular blebs, that are removed from their natural environment, isolated or separated, and are at least 60% free, preferably 75% free, and most preferably 90% free from other components with which they are naturally associated. An “isolated extracellular bleb” is therefore a substantially purified extracellular bleb.

The term “isotype” as used herein refers to the related proteins of particular gene family. Immunoglobulin isotype refers to the distinct forms of heavy and light chains in the immunoglobulins. In heavy chains there are five heavy chain isotypes (alpha, delta, gamma, epsilon, and mu, leading to the formation of IgA, IgD, IgG, IgE and IgM respectively) and light chains have two isotypes (kappa and lambda). Isotype when applied to immunoglobulins herein is used interchangeably with immunoglobulin “class”.

“Isoform” as used herein refers to different forms of a protein which differ in a small number of amino acids. The isoform may be a full-length protein (i.e., by reference to a reference wild-type protein or isoform) or a modified form of a partial protein, i.e., be shorter in length than a reference wild-type protein or isoform.

The term “peptide” is used in its conventional meaning, i.e., as a sequence of amino acids. The peptides are not limited to a specific length of the product. This term also does exclude post-expression modifications of the peptide, for example, glycosylations, acetylations, phosphorylations and the like, as well as other modifications known in the art, both naturally occurring and nonnaturally occurring. In some embodiments, a peptide comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 amino acids, or a range of amino acids that includes or is between any two of the foregoing (e.g., 5 to 50 amino acids).

A “peptide variant” as the term is used herein, is a peptide that typically differs from a peptide specifically disclosed herein in one or more substitutions, deletions, additions and/or insertions. Such variants may be naturally occurring or may be synthetically generated, for example, by modifying one or more of the above peptide sequences of the present disclosure and evaluating one or more biological activities of the peptide as described herein and/or using any of a number of techniques well known in the art.

Modifications may be made in the structure of the peptides of the present disclosure and still obtain a functional molecule that encodes a variant or derivative peptide with desirable characteristics. When it is desired to alter the amino acid sequence of a peptide to create an equivalent, or even an improved, valiant or portion of a peptide of the present disclosure, one skilled in the art will typically change one or more of the codons of the encoding RNA sequence.

For example, certain amino acids may be substituted for other amino acids in a protein structure without appreciable loss of its ability to bind other peptides (e.g., antigens) or cells. Since it is the binding capacity and nature of a protein that defines that protein's biological functional activity, certain amino acid sequence substitutions can be made in a protein sequence, and, of course, its underlying RNA coding sequence, and nevertheless obtain a protein with like properties. It is thus contemplated that various changes may be made in the peptide sequences of the disclosed compositions, or corresponding RNA sequences that encode said peptides without appreciable loss of their biological utility or activity.

In many instances, a peptide variant will contain one or more conservative substitutions. A “conservative substitution” is one in which an amino acid is substituted for another amino acid that has similar properties, such that one skilled in the art of peptide chemistry would expect the secondary structure and hydropathic nature of the peptide to be substantially unchanged.

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 states that the greatest local average hydrophilicity of a protein, as governed by the hydrophilicity of its adjacent amino acids, correlates with a biological property of the protein.

As outlined above, amino acid substitutions are generally therefore based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In another embodiment, variant peptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the peptide.

In certain embodiments, peptide and polynucleotide variants as described herein are peptide or polynucleotide sequences at least 70% identical in to the peptide or polynucleotide sequence they vary from. In other embodiments, peptide and polynucleotide variants as described herein are peptide or polynucleotide sequences that are at least 75, 80, 85, 90, 95, 96, 97, 98 or 99% identical to the peptide or polynucleotide sequence they vary from.

The term “pharmaceutically acceptable” means approved by a regulatory agency of the Federal or a State government or listed in the U.S. Pharmacopeia or other generally recognized pharmacopeia (e.g., Remington's Pharmaceutical Sciences) for use in animals, and more particularly in humans.

Vaccines currently used against infectious diseases including the ongoing COVID-19 pandemic generally consumes a lot of time for their production owing to their complicated and rigorous norms. The vaccines developed for the first strain of COVID-19 have lost efficacy as more highly mutated SARS-CoV-2 strains have come onto the scene. Moreover, their redesign would also be time-consuming, thus increasing the demand for vaccines above supply to an uncontrollable extent. In this regard, it is imperative to start thinking of feasible alternatives. A possible solution to this is by designing a peptide vaccine to enhance the immune system by targeting the antigen presenting cells. For a peptide to be effective in a vaccine to induce cellular immunity, it must first bind within the groove of a major histocompatibility complex (MHC) class I or class II molecule. Second, it must be immunogenic and activate T cells when it is bound by MHC proteins and displayed. Immunogenicity is therefore dependent on the sequence of the peptide displayed. However, a challenge for the design of peptide vaccines is the diversity of human MHC gene alleles that each have specific preferences for the peptide sequences they display, therefore different methods have been used to target different epitopes for peptide vaccine design.

Activating the immune system is reliant on T cells, which orchestrate the types and magnitudes of immune response against an antigen. For efficient T cell activation, a peptide presented with the MHC molecule on an antigen presenting cell (APC) is required along with a costimulatory signal. The corresponding T cell receptor (TCR) and a co-receptor then initiate signal transduction. Therefore, an ideal vaccine should be able to (1) protect not only from the disease but also prevent infection in vaccinated individuals including immunocompromised individuals, (2) process antigenic or antigen-encoding moieties and present desired antigenic peptides by APCs to T cells, (3) elicit long-term immune responses in a desirable fashion with minimal immunizations or booster doses, and (4) have the potential for easy manufacture, storage and accessibility for worldwide vaccination at an affordable cost and limited time. Thus, novel vaccine technologies and further refinement of existing methods and strategies are required to increase the vaccine efficacy. APCs or APC-mimicking materials hold high potential to be an effective, molecularly tunable vaccine platform. In such effort, extracellular vesicles (EVs) have been employed to activate the immune system, often called immunosome. However, EV-based therapeutics have been slow in clinical trials due to their heterogeneity, poor characterization and quantification, and limited mass production. It was found herein that the use of chemicals that can induce cell blebbing were highly efficient in generating high yields of extracellular blebs (EBs) in comparison to production techniques used to produce extracellular vesicles. Moreover, the resulting EBs were homogenous, produced in large quantities, and could be chemically tuned to present desired molecules such as peptides.

Vaccines currently used against the current SARS-CoV-2 generally consume a lot of time for their production owing to their complicated and rigorous process and low vaccine efficacy for a new, mutated strain of SARS-CoV-2. Redesigning a new formulation would also be time-consuming, thus increasing the demand for vaccines to target the mutant strains. An approach that is utilized herein is the use of a computational model evaluating peptide vaccines for eliciting cellular immunity built upon the prediction of peptide presentation by HLA molecules from convalescent patients. The computer-assisted peptide vaccine design used herein targets the SARS-CoV-2 spike protein and its highly mutated regions. More specifically, computer-assisted peptide vaccine design was used to engineer MHC I and MHC II peptides that were expressed in antigen presenting cells, which were then induced to produce extracellular blebs displaying the engineered MHC I and MHC II peptides. This cell-free, cell-mimicking EB platform displays specifically engineered antigens so as to elicit a humoral and cellular immune response to these antigens. As shown in the studies presented herein, the use of said EB platform was successful in generating a humoral and cellular immune response to all known strains of SARS-CoV-2, including those of the COVID-19 omicron lineage.

The disclosure provides innovative vaccine EB preparations that avoid multiple steps used for preparing conventional vaccines. The innovative vaccine EB preparations of the disclosure can be safely and effectively used at low doses with minimal size effects. More importantly, the vaccine EB platform of the disclosure is tunable and can be loaded with a peptide of choice for a directed immune response against a targeted antigenic epitope and quantitatively leveraged immune response. The vaccine EB platform disclosed herein can be used not only for developing emerging vaccines but also studying how immunology plays roles in protecting from and treating a pathogen. While the studies presented herein are directed to SARS-CoV-2, it is clear that the vaccine EB platform of the disclosure can be easily applied to many viral (e.g., influenza) and bacterial pathogens

Current vaccine development efforts are focused on increasing the antibody responses to the spike protein, with a limited focus on T cell immunity and its ability to provide protection against emerging variants. This is due to the receptor binding domain being the main target for neutralizing antibodies produced by B cells. However, antibody titers have shown to be relatively low in COVID-19 recovered individuals. In conditions where antibody titers cannot sufficiently protect against infections, T cell immunity can aid in antibody responses and provide a direct source of T cells for clearing virus-infected cells. For the involvement of T cell immunity in vaccine development, overlapping MHC I and MHC II peptides used in this study can induce both a humoral and cellular immunity in vivo and provide an immune response against the variants of concem.

Recently, DNA vaccine candidates expressing the full-length wild type SARS-CoV-2 spike (S) protein, S1 or S2, showed in mice high levels of specific binding S-specific IgG antibodies and also the activation of T cells and IFN-γ secretion. The full-length S antigen was more potent than the truncated spike (S1 or S2) in inducing neutralizing antibodies and promoting strong T cell responses. Moreover, two COVID-19 vaccines based on modified vaccinia virus Ankara (MVA) vectors expressing the entire SARS-CoV-2 spike (S) protein (MVA-CoV2-S) were evaluated in mice using DNA/MVA or MVA/MVA prime/boost immunizations. Both vaccines induced potent S-specific CD4+ and CD8+ T-cell responses, with a T effector memory phenotype. However, DNA/MVA immunizations elicited higher T-cell immune responses. All vaccine regimens induced high titers of S-specific IgG antibodies. The studies presented herein showed that the prime/boost immunization of the overlapping peptides loaded onto BMDCs and their EBs induced high levels of spike specific antibodies and CD8+ T cell cytotoxicity.

COVID-19 drives substantial T cell activation, with T cell recognition of a large number of SARS-CoV-2-derived peptides. There is also considerable T cell cross recognition in healthy and convalescent individuals. Several studies looking at overlapping peptide pools targeting different regions of SARS-CoV-2 viral proteins have shown a broad range of T cell activation in convalescent COVID-19 patients. Initial analysis of healthy individuals revealed substantial presence of CD4+ and CD8+ T cells that are cross-reactive to SARS-CoV-2 peptides. Studies looking at the cross-reactivity of CD8+ T cells before and after SARS-CoV-2 infection have been investigated only in individual cases. The role of pre-existing T cells in overall immune response and disease outcome is not yet known. A recent study expanded T cells using genome-wide screening and reported cross-reactivity against SARS-CoV epitopes in patients with COVID-19 but not for other coronaviruses.

In the studies presented herein, overlapping MHC I and MHC II peptides that targeted the S protein in the conserved region, were evaluated for CD4+ and CD8+ T cell immunogenicity in convalescent COVID-19 patients. T cell recognition based on—peptides derived from the full SARS-CoV-2 genome and selected based on their predicted HLA-binding capacity were evaluated for their ability to induce CD8+ T cell responses to spike epitopes in 11 patients with COVID-19. Based on their significant values, a few immunodominant and overlapping T cell epitopes were selected. The T cell epitopes used in the studies presented herein are LTDEMIAQY (MHC I) (SEQ ID NO:2) and LPPLLTDEMIAQYTS (MHC II) (SEQ ID NO:1), the former is recognized by CD8 T cells while the latter is recognized by CD4 T cells. The studies presented herein support a role for the importance of T cell immunity against overlapping MHC I and MHC II sequences that are associated with SAR-CoV-2 spike protein to vaccinate against SARS-CoV-2 and its emerging variants of concern.

While the studies presented herein are directed to SARS-CoV-2, it is clear that the vaccine EB platform of the disclosure can be easily applied to many viral (e.g., influenza), fungal and bacterial pathogens. Examples of bacterial pathogens include, but are not limited to, Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi. Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis. Examples of fungal pathogens include, but are not limited to, Absidia corymbifera, Absidia ramose, Achorion gallinae, Actinomadura spp., Ajellomyces dermatididis, Aleurisma brasiliensis, Allersheria boydii, Arthroderma spp., Aspergillus flavus. Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia firfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii. Examples of viral pathogens include, but are not limited to, Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus, KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastern chimpanzee simian foamy virus, Simian foamy virus, Dhori virus, Vientovirus, Human torovirus, Varicella-zoster virus, Chandipura virus, Isfahan virus, and Vesicular stomatitis virus.

Additionally, the vaccine EB platform of the disclosure can be used for noninfectious disease prevention and therapy (e.g., cancer). For example, the molecularly engineered EBs can display engineered MHC I and MHC II peptide sequences that target cancer antigens.

In a particular embodiment, the disclosure provides a vaccine preparation comprising isolated molecularly engineered extracellular blebs. In a certain embodiment, the molecularly engineered extracellular blebs can be isolated from an antigen presenting cells. In another embodiment, the molecularly engineered extracellular blebs present MHC I and MHC II peptide sequences that target specific antigen(s) or a specific epitope thereof. In some embodiments, the molecularly engineered extracellular blebs present MHC I and MHC II peptide sequences that target specific antigen(s). In other embodiments, the molecularly engineered extracellular blebs present MHC I and MHC II peptide sequences that target a specific epitope of targeted antigens. The targeting specific epitopes of targeted antigens provides very important advantages, including but not limited to, (1) all antibodies and T cells are effective, unlike generating polyclonal antibodies and T cells, and (2) activating the immunity against an epitope that is conserved among variants, making a vaccine that consistently works independent of variants.

In particular, the disclosure provides for techniques and methods that provide for high yields of molecularly engineered EBs, in as little as a few hours, producing both micro and nanoscale sized molecularly engineered EBs. For example, use of the blebbing agents described herein can induce the production of EBs in as little as 1-6 h. In a further embodiment, the chemical agent that induces blebbing is a sulfhydryl blocking agent. Examples of sulfhydryl blocking agents include, but are not limited to, mercury chloride, p-chloromercuribenzene sulfonic acid, auric chloride, p-chloromercuribenzoate, chlormerodrin, meralluride sodium, iodoacetmide, paraformaldehyde, dithiothreitol, and N-ethylmaleimide. In a particular embodiment, molecularly engineered EBs are produced from antigen presenting (APC) cells that have molecularly engineered to display antigenic peptides by contacting the cells with a blebbing agent(s) selected from: (1) paraformaldehyde, (2) paraformaldehyde and dithiothreitol, or (3)N-ethylmaleimide. In a further embodiment, molecularly engineered EBs are produced from antigen presenting cells by contacting the antigen presenting cells with a solution comprising paraformaldehyde at of 5 mM, 10 mM, 20 mM, 25 mM, 30 mM, 35 mM, 40 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, or a range that includes any two of the foregoing concentrations (e.g., from 20 mM to 250 mM, from 25 mM to 50 mM, etc.).

In a yet a further embodiment, the solution comprising paraformaldehyde (PFA) further comprises dithiothreitol (DTT) at concentration of 0.2 mM, 0.4 mM, 0.5 mM, 0.6 mM, 0.8 mM, 1 mM, 1.1 mM, 1.2 mM, 1.3 mM, 1.4 mM, 1.45 mM, 1.5 mM, 1.55 mM, 1.6 mM, 1.65 mM, 1.7 mM, 1.75 mM, 1.8 mM, 1.85 mM, 1.9 mM, 1.95 mM, 2.0 mM, 2.1 mM, 2.2 mM, 2.3 mM, 2.4 mM, 2.45 mM, 2.5 mM, 2.55 mM, 2.6 mM, 2.65 mM, 2.7 mM, 2.75 mM, 2.8 mM, 2.85 mM, 2.9 mM, 2.95 mM, 3.0 mM, 3.1 mM, 3.2 mM, 3.3 mM, 3.4 mM, 3.45 mM, 3.5 mM, 3.55 mM, 3.6 mM, 3.65 mM, 3.7 mM, 3.75 mM, 3.8 mM, 3.85 mM, 3.9 mM, 3.95 mM, 4.0 mM, 4.5 mM, 5.0 mM, 5.5 mM, 6.0 mM, 6.5 mM, 7.0 mM, 7.5 mM, 8.0 mM, 8.5 mM, 9.0 mM, 9.5 mM, 10 mM, or any range that includes or is between any two of the foregoing concentrations (e.g., from 1.0 mM to 3 mM, from 1.5 mM to 2.5 mM, etc.). In an alternate embodiment, molecularly engineered EBs are produced from antigen presenting cells by contacting the antigen presenting cells with a solution comprising N-ethylmaleimide (NEM) at concentration of 0.2 mM, 0.4 mM, 0.5 mM, 0.6 mM, 0.8 mM, 1.0 mM, 1.5 mM, 2.0 mM, 2.5 mM, 3.0 mM, 3.5 mM, 4.0 mM, 4.5 mM, 5.0 mM, 5.5 mM, 6.0 mM, 6.5 mM, 7.0 mM, 7.5 mM, 8.0 mM, 8.5 mM, 9.0 mM, 9.5 mM, 10.0 mM, 10.5 mM, 11.0 mM, 11.5 mM, 12 mM, 12.5 mM, 13.0 mM, 13.5 mM, 14.0 mM, 14.5 mM, 15.0 mM, 15.5 mM, 16.0 mM, 16.5 mM, 17.0 mM, 17.5 mM, 18.0 mM, 18.5 mM, 19.0 mM, 19.5 mM, 20.0 mM, or any range that includes or is between any two of the foregoing concentrations (e.g., from 2.0 mM to 20.0 mM, from 2.0 mM to 5.0 mM, etc.). In a further embodiment, the solution comprising PFA; PFA and DTT; or NEN comprises a buffered balanced salt solution. Examples of buffered saline solutions include but are not limited to, phosphate-buffered saline (PBS), Dulbecco's Phosphate-buffered saline (DPBS), Earles's Balanced Salt solution (ICVSS), Hank's Balanced Salt Solution (HBSS), TRIS-buffered saline (TBS), and Ringer's balanced salt solution (RBSS). In a further embodiment, the solution comprising PFA; PFA and DTT; or NEN comprises a buffered balanced salt solution at a concentration/dilution of 0.5×, 0.6×, 0.7×, 0.8×, 0.9×, 1×, 2×, 3×, 4×, 5×, 6×, 7×, 8×, 9×, and 10×, or any range that includes or is between any two of the foregoing concentrations/dilutions, including fractional values thereof.

In a certain embodiment, the disclosure also provides that the molecularly engineered EBs may be collected by any suitable means to separate molecularly engineered EBs from APCs or antigen presenting cell debris. In some embodiments, to isolate molecularly engineered EBs, cells and cell debris can be removed by centrifugation at 100×g to 1000×g for 1, 1.5, 2, 2.5, 3, 3.5, 4, 4.5, 5, 5.5, 6, 6.5, 7, 7.5, 8, 8.5, 9, 9.5, or 10 minutes followed by removal of APCs and antigen cell debris. Molecularly engineered mEBs and nEBs can then be recovered by centrifugation at 10,000×g to 18,000×g for 5, 6, 7, 8, 9, 10, 11, 12, 13, 14, or 15 minutes. Molecularly engineered EBs be further concentrated using concentrators. The size of the molecularly engineered EBs disclosed herein could be controlled by using the isolation methods presented herein.

In a particular embodiment, APCs are phenotypically or genetically modified, so as to express engineered MHC I and MHC II peptide sequences that target specific antigens. The molecularly engineered EBs can then be produced from these genetically modified APCs. The engineered MHC I and MHC II peptide sequences can be taken up by the APCs, or the APCs can be programed to express the engineered MHC I and MHC II peptide sequences. In case of the latter method, various expression vectors can be used including viral vectors. These viral vectors include retroviral vectors, lentiviral vectors, associated adenoviral vectors and adenoviral vectors, among which retroviral vectors and lentiviral vectors are most widely used. Viral vectors are capable of ensuring stable expression of the engineered MHC I and MHC II peptide sequences. A non-viral Sleeping Beauty (SB) transposon system may also be used to generate stable engineered MHC I and MHC II peptide expression but without the risks associated with viral vectors.

The disclosure further provides that the vaccine preparations comprising the molecularly engineered EBs may be used (1) in combination with other agents or molecules, and/or (2) loaded with other agents or molecules, such as biological molecules, therapeutic agents (e.g., antibiotics), adjuvants, etc. In a particular embodiment, the vaccine preparations disclosed herein further comprise or are used in combination with an adjuvant that creates a stronger immune response in subjects receiving the vaccine. Examples of such adjuvants include, but are not limited to, aluminum salts (e.g., aluminum hydroxide, aluminum phosphate, and aluminum potassium sulfate), AS04, MF59, ASO1_{B}, and CpG 1018. It should be further noted that the molecularly engineered EBs may be loaded with the other agents or molecules, such as adjuvants.

The molecularly engineered EBs may be loaded with the other agents or molecules via direct membrane penetration, chemical labeling and conjugation, electrostatic coating, adsorption, absorption, electroporation, or any combination thereof. Further, molecularly engineered EBs produced in accordance with certain embodiments of the disclosure may undergo multiple loading steps, such that other agents or molecules may be introduced to APCs prior to blebbing, while additional other agents or molecules may be loaded during or after blebbing. Additionally, molecularly engineered EBs may be loaded with the other agents or molecules during blebbing, and further loaded with other agents or molecules after blebbing. In a further embodiment, the molecularly engineered EBs may be loaded with other agents or molecules as defined above by incubating APCs or molecularly engineered EBs with the other agents or molecules having the concentration of 25 pg/mL, 50 pg/mL, 100 pg/mL, 200 pg/mL, 300 pg/mL, 400 pg/mL, 500 pg/mL, 600 pg/mL, 700 pg/mL, 800 pg/mL, 900 pg/ml, 1 ng/mL, 10 ng/mL, 100 ng/mL, 1 pg/mL, 10 pg/mL or any range that includes or is between any two of the foregoing concentrations. Additionally, the incubation may occur for 1 hour, 2 hours, 3 hours, 4 hours, 5 hours, 6 hours, 12 hours, 24 hours, 48 hours, or any range that includes or is between any two of the foregoing time points.

The disclosure further provides for specified modes of administration for administering the vaccine preparations comprising the molecularly engineered EBs disclosed herein. In one embodiment, a vaccine preparation comprises the molecularly engineered EBs and a pharmaceutically acceptable carrier. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the composition and is compatible with administration to a subject, for example a human. Such compositions can be specifically formulated for administration via one or more of a number of routes, such as the routes of administration described herein. Supplementary active ingredients also can be incorporated into the compositions. When an agent, formulation or pharmaceutical composition described herein, is administered to a subject, preferably, a therapeutically effective amount is administered. As used herein, the term “therapeutically effective amount” refers to an amount that result in an improvement or remediation of the condition.

The disclosure further provides for the use of a vaccine preparation comprising molecularly engineered EBs for vaccinating a subject. Suitable methods of administering a vaccine preparation described herein to a patient include by any route of in vivo administration that is suitable for delivering molecularly engineered EBs to a patient. Examples of modes of administration include, but are not limited to, intravenous administration, intertumoral administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), nasal, oral, pulmonary administration, impregnation of a catheter, and direct injection into a tissue.

Intravenous, intraperitoneal, and intramuscular administrations can be performed using methods standard in the art. Aerosol (inhalation) delivery can also be performed using methods standard in the art (see, for example, Stribling et al., Proc. Natl. Acad. Sci. USA 189: 11277-11281, 1992, which is incorporated herein by reference in its entirety). Oral delivery can be performed by complexing a vaccine preparation of the present invention to a carrier capable of withstanding degradation by digestive enzymes in the gut of an animal. Examples of such carriers include plastic capsules or tablets, such as those known in the art.

The appropriate dosage and treatment regimen for the vaccine preparations described herein will vary with respect to the needed vaccination schedule of the subject. In certain cases, only one vaccine preparation may need to be administered to a subject to bring about effective immunity to a pathogen. In other cases, one or more booster shots of the vaccine preparation may be needed. In such a case, the one or more booster shots may have the same dose of the biological engineered EBs or be of a lower dose. For multiple doses of the vaccine preparation, they may be administered a week or more apart.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more vaccine EB preparations described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific application. The label can also indicate directions for use of the contents, such as in the methods described herein.

The disclosure further provides that the devices, platforms, systems, devices and methods described herein can be further defined by the following aspects (aspects 1 to 44):
1. A vaccine preparation comprising:
isolated or purified extracellular blebs (EBs) that present engineered MHC I and MHC II peptides that target specific antigen(s) or a specific epitope(s) from a pathogen or a disease; wherein the EBs are isolated or purified from an antigen presenting cell.
2. The vaccine preparation of aspect 1, wherein the antigen presenting cell is selected from a dendritic cell, a macrophage, and a B-Cell.
3. The vaccine preparation of aspect 2, wherein the antigen presenting cell is a dendritic cell.
4. The vaccine preparation of any one of the proceeding aspects, wherein the antigen presenting cell presents the engineered MHC I and MHC II peptides that target specific antigen(s) or a specific epitope(s).
5. The vaccine preparation of any one of the proceeding aspects, wherein the pathogen is selected from a fungus, a virus, or a bacterium, and the disease is cancer or an immune disease.
6. The vaccine preparation of aspect 5, wherein the pathogen is a bacterium selected from the following: Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira welii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.
7. The vaccine preparation of aspect 5, wherein the pathogen is a fungus selected from the following: Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya funigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.
8. The vaccine preparation of aspect 5, wherein the pathogen is a virus selected from the following: Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus, KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastern chimpanzee simian foamy virus, Simian foamy virus, Dhori virus, Vientovirus, Human torovirus, Varicella-zoster virus, Chandipura virus, Isfahan virus, and Vesicular stomatitis virus.
9. The vaccine preparation of aspect 8, wherein the Human SARS coronavirus is SARS-CoV-2, and/or a variant thereof.
10. The vaccine preparation of aspect 9, wherein the specific antigen(s) comprises a peptide sequence for a portion of the spike protein from SARS-CoV-2 and/or a variant thereof.
11. The vaccine preparation of any one of the proceeding aspects, wherein the engineered MHC I peptide comprises the sequence of SEQ ID NO:2, SEQ ID NO:3, SEQ ID NO:4, SEQ ID NO:5 or SEQ ID NO:6.
12. The vaccine preparation of aspect 11, wherein the engineered MHC I peptide comprises the sequence of SEQ ID NO:2.
13. The vaccine preparation of aspect 11, wherein the engineered MHC I peptide has a sequence that consists essentially of SEQ ID NO:2.
14. The vaccine preparation of any one of the proceeding aspects, wherein the engineered MHC II peptide comprises the sequence of SEQ ID NO:1.
15. The vaccine preparation of aspect 14, wherein the engineered MHC II peptide has a sequence that consists essentially of SEQ ID NO:1.
16. The vaccine preparation of any one of the proceeding aspects, wherein the vaccine preparation further comprises an adjuvant.
17. The vaccine preparation of aspect 16, wherein the adjuvant is selected from aluminum hydroxide, aluminum phosphate, aluminum potassium sulfate, AS04, MF59, ASOI_{B}, and CpG 1018.
18. The vaccine preparation of any one of the proceeding aspects, wherein the vaccine preparation is formulated for intramuscular delivery, subcutaneous delivery, intradermal, or intranasal delivery.
19. The vaccine preparation of any one of the proceeding aspects, wherein the vaccine preparation is administered as a single dose, or as a primary dose with one or more follow up dose(s).
20. The vaccine preparation of aspect 19, wherein the vaccine preparation is administered as a primary dose with one or more follow up dose(s), where there is at least 7 days between the administration of each dose.
21. A method of making a vaccine preparation of any one of the proceeding aspects, the method comprising:
treating an antigen presenting cell that displays engineered MHC I and MHC II peptide sequences that target specific antigen(s) or a specific epitope(s) from a pathogen with a blebbing agent;
isolating EBs from the antigen presenting cell;
preparing a vaccine preparation comprising the isolated EBs.
22. The method of aspect 21, wherein the blebbing agent comprises paraformaldehyde or N-ethylmaleimide.
23. The method of aspect 21 or aspect 22, the method further comprising:
engineering MHC I and MHC II peptide sequences that target specific antigen(s) or a specific epitope(s) from a pathogen using a computational model of peptide vaccines for eliciting cellular immunity based upon the prediction of peptide presentation by HLA molecules from patients that were infected by the pathogen; and
introducing or expressing the engineered MHC I and MHC II peptide sequences into an antigen presenting cell.
24. The method of any one of aspects 21 to 23, wherein the pathogen is selected from a fungus, virus, or bacterium; and the disease is cancer or an immune disease.
25. The method of aspect 24, wherein the pathogen is a bacterium selected from the following: Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae. Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.
26. The method of aspect 24, wherein the pathogen is a fungus selected from the following: Absidia corymbifera, Absidia ramose, Achorion gallinae, Actinomadura spp., Ajellomyces dermatididis, Aleurisma brasiliensis, Allersheria boydli, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattia, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.
27. The method of aspect 24, wherein the pathogen is a virus selected from the following: Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus, KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastern chimpanzee simian foamy virus, Simian foamy virus, Dhori virus, Vientovirus, Human torovirus, Varicella-zoster virus, Chandipura virus, Isfahan virus, and Vesicular stomatitis virus.
28. The method of aspect 27, wherein the Human SARS coronavirus is SARS-CoV-2, and/or a variant thereof.
29. The method of any one of aspects 21 to 28, wherein the engineered MHC I peptide comprises the sequence of SEQ ID NO:2, SEQ ID NO:3, SEQ ID NO:4, SEQ ID NO:5 or SEQ ID NO:6.
30. The method of aspect 29, wherein the engineered MHC I peptide comprises the sequence of SEQ ID NO:2.
31. The method of aspect 30, wherein the engineered MHC I peptide has a sequence that consists essentially of SEQ ID NO:2.
32. The method of any one of aspects 21 to 31, wherein the engineered MHC II peptide comprises the sequence of SEQ ID NO:1.
33. The method of aspect 32, wherein the engineered MHC II peptide has a sequence that consists essentially of SEQ ID NO:1.
34. A method for vaccinating a subject against a pathogen, comprising:
administering to the subject one or more doses of the vaccine preparation of any one of aspects 1 to 20.
35. The method of aspect 34, wherein the pathogen is selected from a fungus, a virus, or a bacterium.
36. The method of aspect 35, wherein the pathogen is a bacterium selected from the following: Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningilidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis. Yersinia enterocolitica, and Yersinia pseudotuberculosis.
37. The method of aspect 35, wherein the pathogen is a fungus selected from the following: Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophytonfloccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.
38. The method of aspect 35, wherein the pathogen is a virus selected from the following: Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus, KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastern chimpanzee simian foamy virus, Simian foamy virus, Dhori virus, Vientovirus, Human torovirus, Varicella-zoster virus, Chandipura virus, Isfahan virus, and Vesicular stomatitis virus.
39. The method of aspect 38, wherein the Human SARS coronavirus is SARS-CoV-2, and/or a variant thereof.
40. The method of any one of aspects 34 to 39, wherein the vaccine preparation comprises an adjuvant or is co-administered with an adjuvant.
41. The method of aspect 40, wherein the adjuvant is selected from aluminum hydroxide, aluminum phosphate, aluminum potassium sulfate, AS04, MF59, ASO1B, and CpG 1018.
42. The method of any one of aspects 34 to 42, wherein the vaccine preparation is administered intramuscularly, subcutaneously, intradermally, or intranasally to the subject.
43. The method of any one of aspects 34 to 42, wherein the vaccine preparation is administered to the subject as a single dose, or as a primary dose with one or more follow up dose(s).
44. The method of aspect 43, wherein the vaccine preparation is administered to the subject as a primary dose with one or more follow up dose(s), where there is at least 7 days between the administration ofeach dose.

### EXAMPLES

In order that the present disclosure may be more fully understood, the following examples are set forth. It should be understood that these examples are for illustrative purposes only and are not to be construed as limiting this disclosure in any manner.

### Materials

| Mouse | Vendor |
|---|---|
| C57BL/6, female, 6-8 weeks old | Charles River Laboratories |

| Peptide | Synthesis (Vendor) |
|---|---|
| MHC I (LTDEMIAQY) | Anaspec |
| MHC II (LPPLLTDEMIAQYTS) | Anaspec |

| | Vendor and Catalogue |
|---|---|
| Protein and pseudovirus | number |
| Spike protein | Sinobiological 40150-D003 |
| SARS-CoV-2 spike alpha pseudovirus | BPS bioscience (78112) |
| SARS-CoV-2 spike delta pseudovirus | BPS bioscience (78216) |
| SARS-CoV-2 spike omicron pseudovirus | BPS bioscience (78394) |

| Chemicals | Vendor | Catalogue number |
|---|---|---|
| RPMI | Fisher | SH30027.01 |
| DMEM | Fisher | 11965118 |
| Fetal bovine serum (FBS) | Fisher | MT35011CV |
| Penicillin-streptomycin | Fisher | 15-140-122 |
| Red blood cell lysis buffer | Fisher | 50-112-9751 |
| rmGM-CSF | R&D Systems | 415-ML-020 |
| SIINFEKL | Invivogen | OVA 257-264 |
| Lipopolysaccharide (LPS) | Sigma Aldrich | L4516-1MG |
| Paraformaldehyde (PFA) | Fisher | AC416780250 |
| 1 × DPBS | Fisher | MT-21-030-CV |
| Trypan blue | Fisher | MT25900CI |
| ELISA coating buffer | Fisher | |
| ELISA wash buffer | Fisher | |

| Cell staining supplies | Vendor | Catalogue number |
|---|---|---|
| Propidium iodide (PI) | Biolegend | 421301 |
| CellTrace Blue | ThermoFisher | C34568 |
| Yo-Pro-1 | ThermoFisher | Y3603 |

| | | Catalogue |
|---|---|---|
| Cell culture equipment | Vendor | number |
| 5 mL sterile syringe | VWR | 309646 |
| 25-gauge needle | Fisher | 14-817-168 |
| 40 μm sterile cell strainer | Fisher | 07-201-430 |
| Eppendorf tubes, 1.5 mL | ThermoFisher | 90410 |
| Amicon centrifugal filter (MWCO 100 | Fisher | UFC910024 |
| kDa) | | |
| 5 cm² cell culture dishes | Fisher | 08-772B |
| 100 cm² cell culture dishes | Fisher | 08-772E |
| 96 well plate (flat bottom) | Fisher | 07-200-90 |
| 96 well plate (round bottom) | Fisher | FB012932 |

| Equipment | Vendor |
|---|---|
| Incubator | ThermoFisher Scientific, USA |
| Hemocytometer grids | Hausser Scientific, Horsham, USA |
| Centrifuges | ThermoFisher Scientific, USA |
| Vortex | Scientific Industry, NY, USA |
| Heat block | ThermoFisher Scientific, USA |
| Guava EasyCyte flow cytometer | Luminex, USA |
| Fortessa flow cytometer | BD Biosciences, USA |

| Software | Source |
|---|---|
| Computational analysis | Ahmed |
| FlowJo | FlowJo |
| GraphPad Prism (Version 8) | GraphPad Prism |
| Microsoft Office (Excel, Word, PowerPoint) | Microsoft office 365 |

EL4 spike celllineproduction. Mouse T cell lymphoma EL4 cells were cultured in DMEM supplemented with 10% FBS, 1% Penicillin-Streptomycin. EL4 cells were plated at a density of 5×10⁵ per well in a 6-well plate and were transduced with 2×10⁶ TU/well of SARS-CoV-2 spike protein (SPK alpha)-encoding lentivirus (BPS bioscience, San Diego, CA) in the presence of 5 μg/mL of polybrene (Thermo Fisher Scientific). After 72 h of transduction, the transduced cells were selected by 0.5 μg/mL of puromycin (Thermo Fisher Scientific) for 2 weeks. The SPK expression in the resulting EL4 cells was analyzed by flow cytometry after staining with anti-SPK S1 primary antibody (BPS Bioscience) and FITC-conjugated goat anti-human IgG secondary antibody (Thermo Fisher Scientific). EL4 spike expressing cells were incubated at 37° C. with 5% CO₂ and 100% humidity.

Bone marrow derived dendritic cell isolation and culture. C57BL/6 mice (6-12-week-old female) were euthanized by use of CO₂ followed by cervical dislocation. Hind quarter femurs were harvested from the euthanized mice and the bone marrow was flushed out using a 25-gauge needle with RPMI media containing 10% FBS and 1% Penicillin-Streptomycin into a 40 μm sterile cell strainer. Bone marrow was homogenized and flushed through the cell strainer. Cells were collected cells and centrifuged at 300×g for 10 min. The supernatant was aspirated, and the cells were re-suspended in 1×RBC lysis buffer and incubated for 10 min at room temperature. After which, RPMI culture media was added, and the resulting mixture was centrifuged at 300×g for 10 min. The supernatant was aspirated, and the cell pellets were re-suspended in DC differentiation media (RPMI supplemented with 10% FBS and 1% Penicillin-Streptomycin and 20 ng/mL of mGM-CSF) and plated at a density of 1 femur cell pellet/100 cm² cell culture dish. The DC differentiation media was supplemented with 10 mL of a freshly prepared media after 2 days. 5 days post differentiation, immature DCs were activated in presence of LPS (20 ng/mL) to induce maturation into mature DCs.

Optimizing peptide loading on dendritic cell lines. To determine the concentration for peptide loading on the dendritic cell surface, competition binding to MHC I (H2Kb) with SIINFEKL peptide (SEQ ID NO:7) was analyzed by flow cytometry using a commercial SIINFEKL/H2Kb-specific antibody. SIINFEKL, was pre-loaded onto the dendritic cells cell surface at a concentration of 1 μg/mL. To which, different concentrations of the LTDEMIAQY peptide (SEQ ID NO:2), ranging from 5 μg/mL to 1 μg/mL, was loaded onto the dendritic cells to determine the peptide replacement. The peptides were successful in replacing the pre-loaded SIINFEKL at a concentration of 1 μg/mL.

Loading DCs with MHC I and MHC II T cell peptides. After 5 days of differentiation, loosely adherent immature DCs were collected by gently rinsing the plates with a serological pipette and centrifuged at 300×g for 10 min. Collected DCs were incubated with 1 μg/mL LTD-MHC I peptide and 100 μg/mL LTD-MHC II peptide in RPMI complete media for 1 h. MHC I and MHC II peptide-loaded BMDCs were then incubated in RPMI media for 24 h to prepare MHC I and MHC II peptide-loaded immature DCs (iDCs), or with RPMI media supplemented with 20 ng/mL lipopolysaccharide (LPS) for 24 h in order to prepare MHC I and MHC II peptide-loaded mature DCs (mDCs). All BMDCs used in the study were loaded with MHC I and MHC II-peptides.

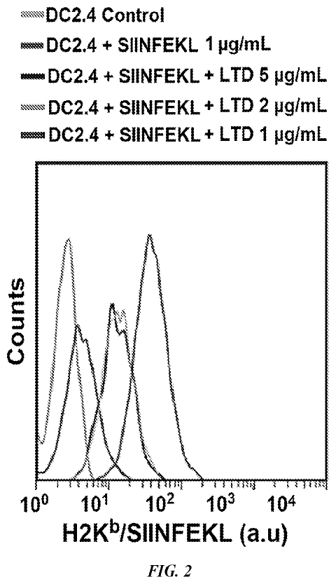

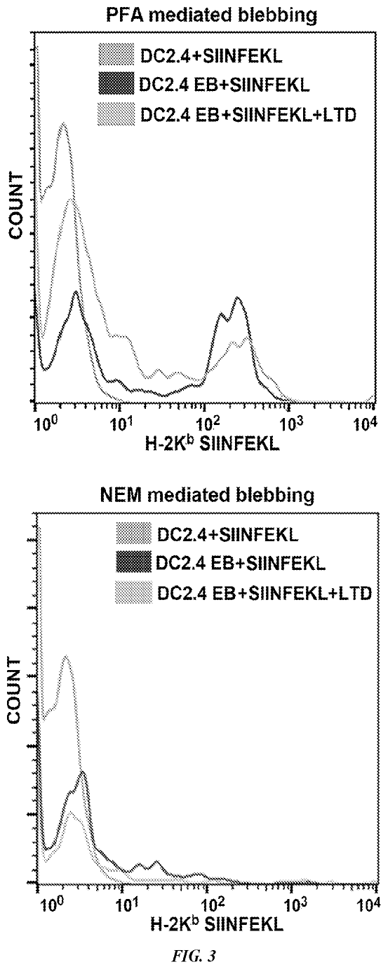

Comparing production and stability of chemically induced blebs expressing MHC 1peptide using PFA vs. NEM. Dendritic cells were loaded with SIINFEKL and SIINFEKL+LTDEMIAQY and incubated with 2 mM N-ethylmaleimide (NEM) in 1×DPBS for 8 h, or with 25 mM paraformaldehyde for 24 h, at 37° C. in presence of 5% CO₂ to induce extracellular bleb production. After removal of cell debris by centrifugation at 1000×g for 5 minutes, the DC-EBs were purified or isolated by centrifugation of the supernatant at 16.100×g for 10 min. This step was repeated until cell debris was not visible. The resulting isolated or purified peptide loaded EBs were analyzed for stability and the ability to maintain peptide presentation after blebbing (see FIG. 2). The blebbing agent paraformaldehyde, PFA, immobilized the cell surface molecules such as antigenic peptides on MHC molecules and did not disrupt peptide presentation on the cell surface during the blebbing process, while NEM-mediated blebbing disrupted peptide presentation.

Blebbing buffer for peptide-loaded BMDCs. Paraformaldehyde (PFA, 4 g) was added to 50 mL DI water, stirred continuously and heated to 60° C., then immediately adjusted with iN NaOH until the solution turned from milky white to clear. 50 mL of DI water was added to bring the final volume to 100 mL, making a 4% (w/vol) PFA solution. The pH of the solution was adjusted to ˜7 by addition of a IN HCl solution. This PFA stock solution was sterilized using a 0.22 μM filter and stored at 4° C. The 25 mM PFA blebbing buffer was prepared by diluting 180 μL of PFA stock solution in 10 mL 1×DPBS, immediately before use.

Production of MHC I and MHC I-presenting DC-EBs. After 24 h of incubation with MHC I and MHC II-peptides, immature DCs or mature DCs were collected and centrifuged at 300×g for 10 min. The cell pellet was re-suspended in 1×DPBS and centrifuged at 300×g for 10 min, which was repeated 3 times to remove proteins, cell debris, and other impurities. The cells were then incubated in PFA blebbing buffer overnight at 37° C. with 5% CO₂to induce the production of immature DC-EBs from immature DCs (imDCs), and the production of mature DC-EBs from mature DCs (mDCs). To isolate micro-scale DC-EBs, the supernatant was collected after removing cell debris by centrifugation at 300×g for 10 min, followed by centrifugation at 16,100×g for 10 min. The collected DC-EBs were further rinsed 3 times with 1×DPBS via repeated centrifugation to remove any residual blebbing reagents and cell debris. The DC-EB pellets were finally resuspended in I×DPBS and confirmed to be free of cells and debris by microscopy.

Animal vaccination studies. All animal work was reviewed and approved by the UCI Institutional Animal Care and Use Committee. Female 7-10-week-old C57BL/6 mice (Charles River Laboratories, Wilmington, MA) were subcutaneously injected with 100 μL of 1×DPBS, free MHC I and MHC II peptides, 2.5×10⁵ MHC I and MHC II-peptide loaded BMDCs, or MHC I and MHC II-peptide loaded BMDC EBs at an equivalent surface area to MHC I and MHC II-peptide loaded BMDCs. The mice received a priming injection on Day 0 and a booster injection on Day 14. Immediately before and 10 days after the booster injection (Day 14 and 24), blood was collected into heparinized micro-capillary tubes from the saphenous vein.

In vivo spike ELISA analysis and virus neutralization assay from mice sera. Sera collected as explained above were tested for SARS-CoV-2 spike proteins binding by ELISA. Briefly, recombinant SARS-CoV-2 spike protein was coated at 2 μg in 100 μL coating buffer per well of a 96-well plate overnight. The plates were blocked by adding 100 μL per well of blocking buffer consisting of 5% (w/v) non-fat dry milk in DPBS containing 0.05% (w/v) Tween-20 and incubated at room temperature for 2 h. The plate was then rinsed three times using an ELISA wash buffer. The plasma samples were diluted 20-fold in 150 μL 1×ELISA diluent buffer and were added to the wells and incubated for 2 h at room temperature. The plates were washed three times using an ELISA wash buffer, and the detection antibody (mouse IgG-HRP [H+L], Waltham, MA) was added at the manufacture's recommended concentration followed by an additional 1 h incubation at room temperature. After the plate was washed five times, 100 μL of a TMB substrate solution (Thermo Fisher Scientific) was added to each well. The plates were incubated for 15 min at room temperature. The reaction was stopped by the addition of 100 μL ELISA stop solution per well and the absorbance was measured at 450 nm and 570 nm using a plate reader (SpectraMax Plus, Molecular Devices, USA). The absorbance reading at 570 nm was subtracted by that at 450 nm for optical correction. The antibody concentration in the plasma samples were quantified by using calibration curve with known concentrations of a standard. For the virus neutralization assay, 100 μL of serially diluted plasma was incubated with 100 μL of 1×10⁴ GFP-expressing lentivirus pseudotyped with SARS-CoV-2 spike alpha, delta or omicron protein in DMEM media supplemented with 10% FBS and 1% penicillin-streptomycin for 1 h at 37° C. After which, 1×10⁴ ACE2 expressing 293T (293T ACE2) cells were added and the samples were incubated for 48 h. The cells were analyzed for GFP expression using flow cytometry. The relative transduction inhibition was determined by mean GFP fluorescence intensity (MFI).

Specific lysis of E.G7-OVA cells by splenocytes and CD8 T cell proliferation assay. C57BL/6 mice (female, 6-8-week-old) were vaccinated (3 mice per treatment group) and sacrificed 10 days after the booster vaccinations. The spleens were harvested in a sterile environment and placed in 5 cm² cell culture dishes in 5 mL splenocyte media. The collected spleens were macerated through a 40 μm sterile tissue strainer into a conical tube. The cells were collected with a serological pipette and centrifuged at 350×g for 10 min. The cells were then incubated with ice-cold 1×RBC lysis buffer to deplete the RBCs. EL4-spike cells labeled with CellTrace Blue were plated in round bottom 96 well plates at an E:T (splenocyte: E.G7-OVA) ratio of 25:1 for 4 h at 37° C. with 5% CO₂ and 100% humidity. Plates were centrifuged at 300×g for 10 min; cells were washed once in 1×PBS and incubated with 1 μL/mL Yo-Pro-1 for 15 min on ice. After rinsing three times with 1×PBS, the cells were analyzed by flow cytometry.

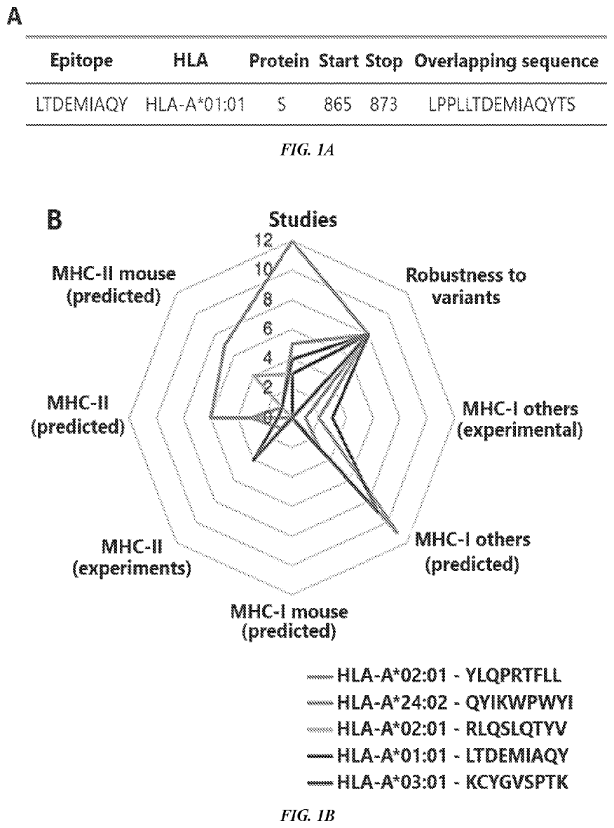

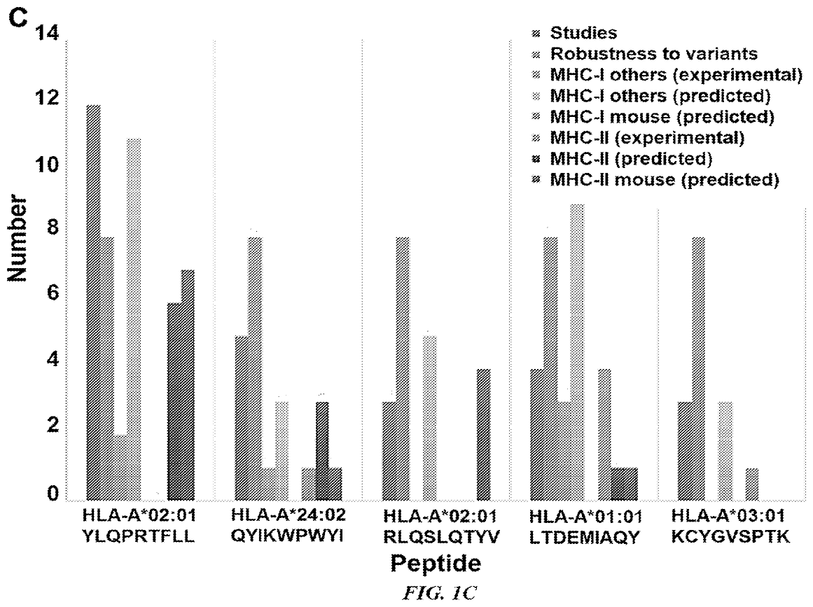

In silico approach for conserved T cell epitope targeting SARS-CoV-2 S protein. Activating B-cells and T-cells to target the SARS-CoV-2 spike protein is a promising approach to target SARS-CoV-2 and its emerging variants. Hence, in this study, the spike protein sequence of the SARS-CoV-2 was chosen as the main subject in the design of the multi-epitope vaccine (see FIG. 1).

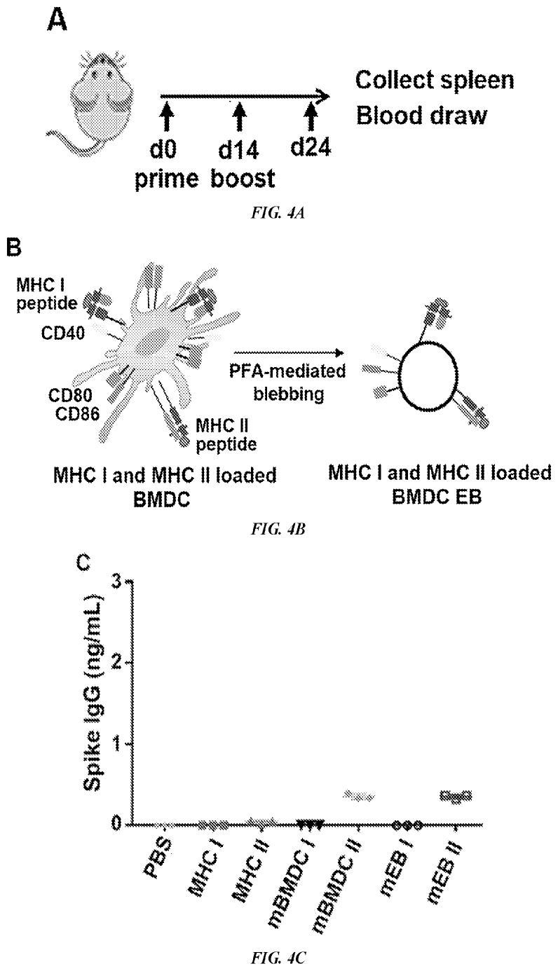

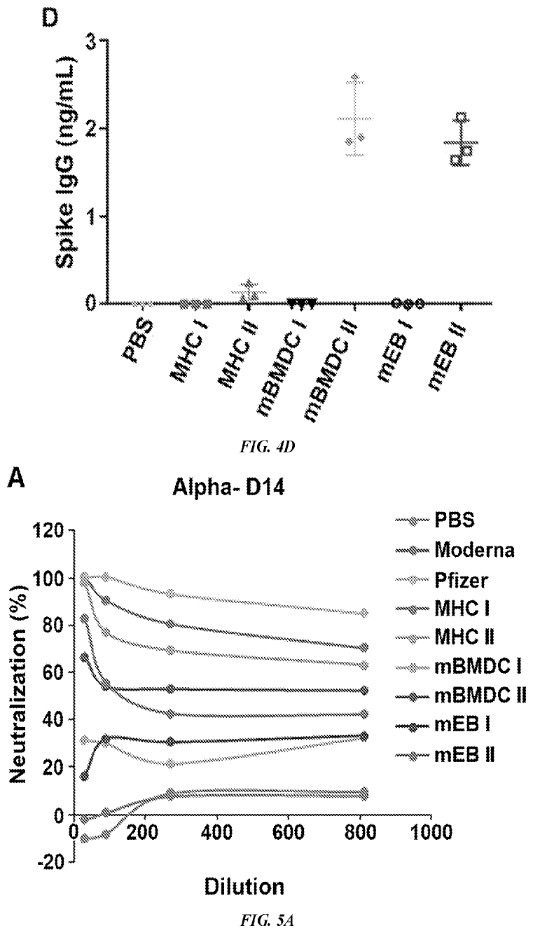

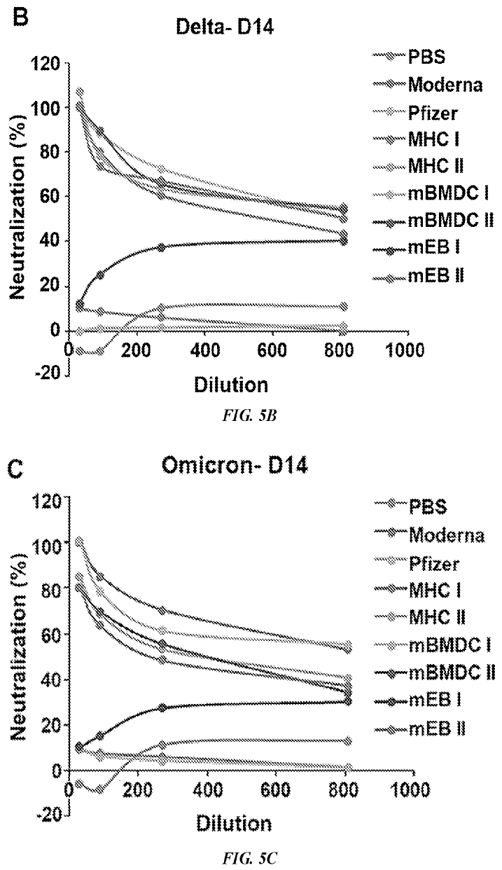

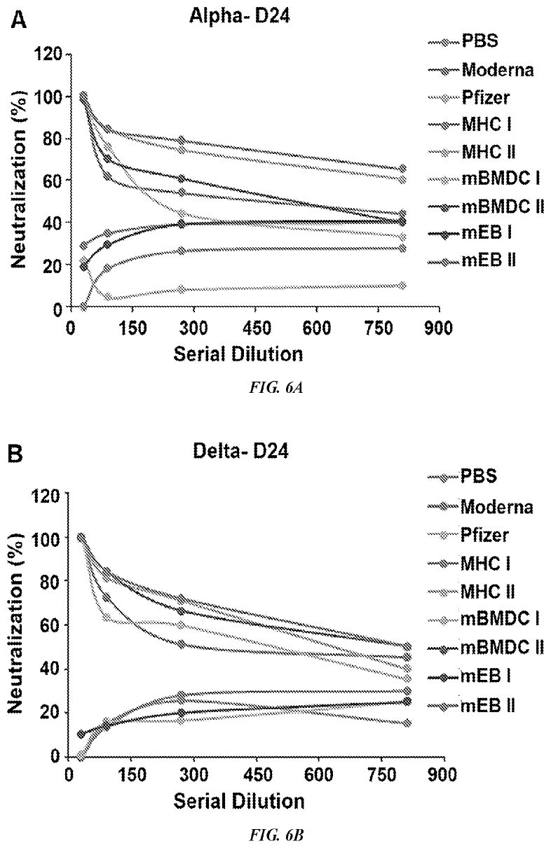

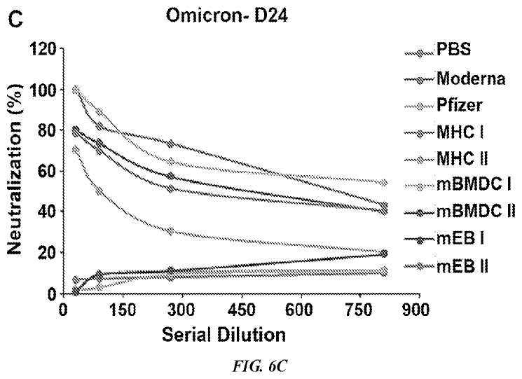

Immunogenicity of peptide loaded extracellular blebs. Based on the computational approach, MHC I and MHC II overlapping peptide sequences that target the SARS-CoV-2 S protein were obtained. The targeting MHC I and MHC II peptides allowed for antigen-presenting cells (APCs) to simultaneously uptake peptide antigens to induce potent and specific antigen-specific immune responses. To first evaluate the immunogenicity of MHC I and MHC II peptide loaded EBs, C57BL/6 mice were injected PBS, free MHC I (1 μg/mL) and MHC II peptide (100 μg/mL), 2.5×10⁵ MHC I and MHC II peptide loaded BMDCs (parental cells) and MHC I and MHC II peptide loaded DC EBs at an equivalent surface area of 2.5×10⁵ of the parental cells. All groups were subcutaneously injected 14 days apart at an equivalent amount of the parental cells by surface area for a total of 2 doses (see FIG. 4A). The serum samples obtained 14 and 24 days after the prime and booster vaccination showed an antibody response against spike protein, with IgG-type antibody titers of up to 0.5-fold and 2-fold on day 14 and 24 respectively for the MHC II-peptide loaded groups (see FIGS. 4C and 4D). However, the antisera obtained from the mice exhibited ineffective antibody production against the delta and omicron variants which was likely due to the fact that the peptides are located in the conserved region and not in the receptor binding domain (RBD) region. There are reports showing that linear peptides have difficulty in inducing neutralizing antibodies in mice. Thus, further immunogenicity of the MHC I and MHC II-peptide loaded BMDCs and their respective EBs were assessed for neutralization assay using the pseudotyped-spike virus targeting the alpha, delta or omicron strain of Covid. Although no spike antibody was observed in both the delta and omicron samples, the sera showed higher neutralizing antibody responses in both day 14 and 24 for the MHC II-peptide loaded groups (see FIG. 5 and FIG. 6). These results demonstrate that the MHC II-peptide loading directly targeted APCs and the robust antibody immunity indicated the potential for a peptide vaccine. Vaccination of mice with the MHC II peptide loaded BMDCs and their respective EBs elicited CD4 T cells, as well as B cell responses, resulting in the formation of antibodies that neutralized pseudotyped SARS-CoV-2 spike proteins.

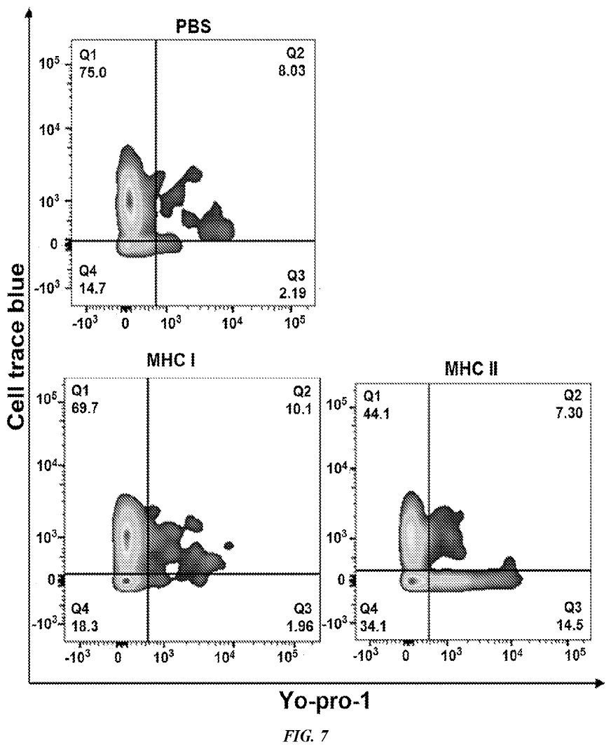

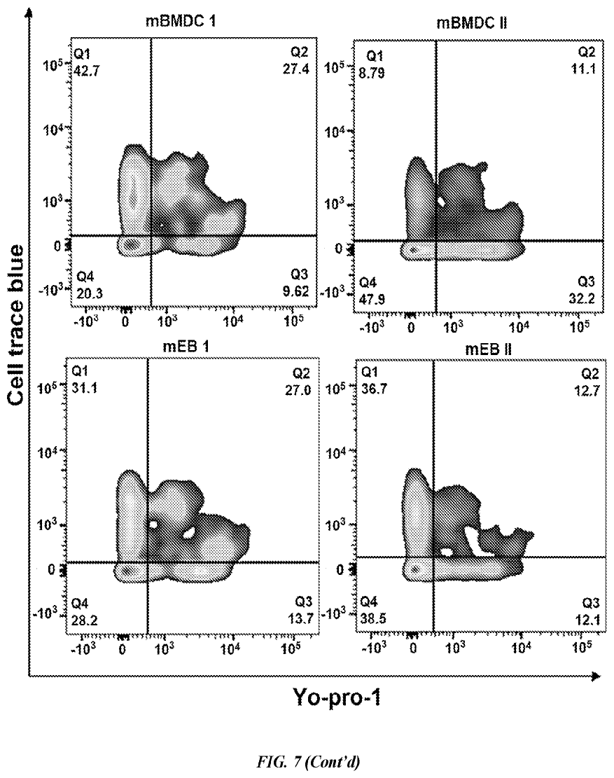

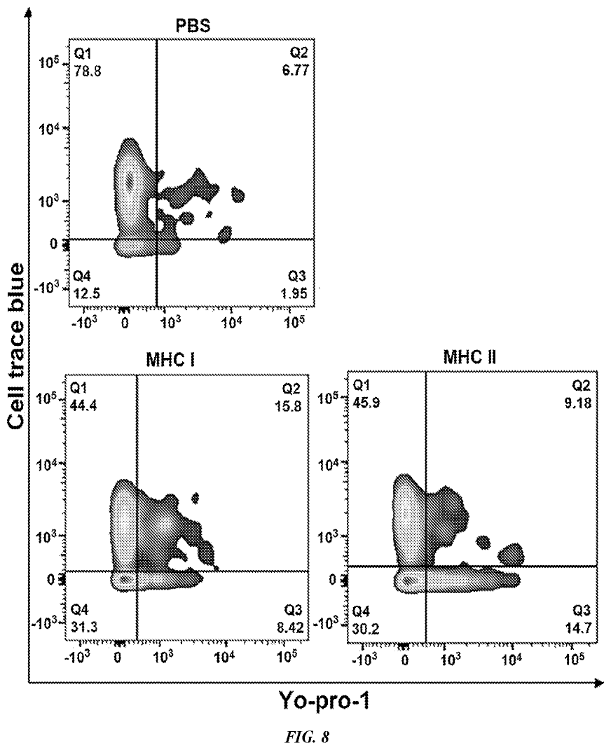

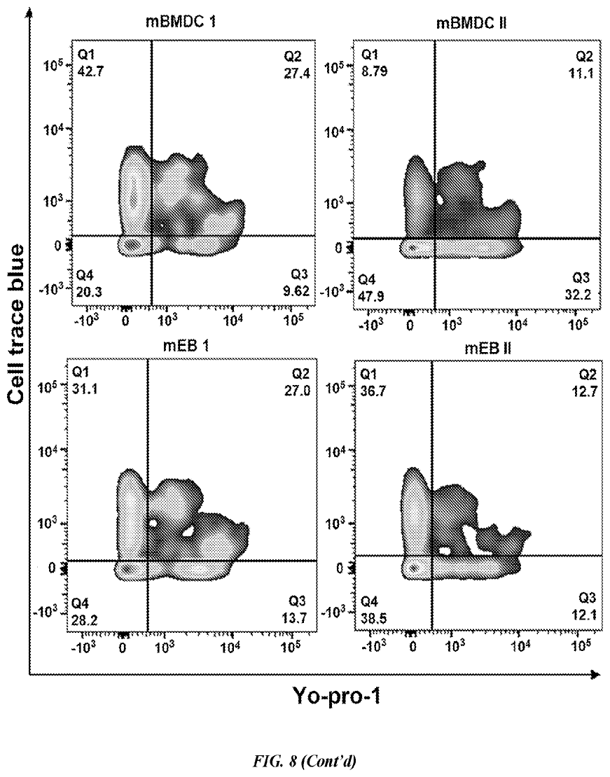

Cellular immunity in peptide loaded extracellular blebs. Cellular immunity, particularly exerted by T cells, is an important factor for protection against memory of SARS-CoV-2 infections, it was next determined the T cell response in animals immunized with PBS, free MHC I (1 μg/mL) and MHC II peptide (100 μg/mL), 2.5×10⁵ MHC I and MHC II peptide loaded BMDCs and MHC I and MHC II peptide loaded DC EBs at an equivalent surface area of 2.5×10⁵ of the parental cells. Splenocytes harvested 10 d post immunization were stimulated with MHC I peptides overnight prior to CTL specific lysis analysis by flow cytometry. Whole spleens were isolated and analyzed for specific lysis in EL4-spike expressing cells and at E:T ratio of 25:1, approximately 16% (see FIG. 7) and 27% (see FIG. 8) of EL4-spike expressing cells. The EL4-spike expressing cells were lysed by splenocytes in the MHC I peptide loaded BMDCs and EBs respectively.

A number of embodiments have been described herein. Nevertheless, it will be understood that various modifications may be made without departing from the spirit and scope of this disclosure. Accordingly, other embodiments are within the scope of the following claims.

1. A vaccine preparation comprising:
isolated or purified extracellular blebs (EBs) that present engineered MHC I and MHC II peptides that target specific antigen(s) or a specific epitope(s) from a pathogen or a disease,
wherein the EBs are isolated or purified from an antigen presenting cell.

2. The vaccine preparation of claim 1, wherein the antigen presenting cell is selected from a dendritic cell, a macrophage, and a B-Cell.

3. The vaccine preparation of claim 2, wherein the antigen presenting cell is a dendritic cell.

4. The vaccine preparation of claim 1, wherein the antigen presenting cell presents the engineered MHC I and MHC II peptides that target specific antigen(s) or a specific epitope(s).

5. The vaccine preparation of claim 1, wherein the pathogen is selected from a fungus, a virus, or a bacterium, and the disease is cancer or an immune disease.

6. The vaccine preparation of claim 5, wherein the pathogen is a bacterium selected from the following: Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

7. The vaccine preparation of claim 5, wherein the pathogen is a fungus selected from the following: Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.

8. The vaccine preparation of claim 5, wherein the pathogen is a virus selected from the following: Human coronavirus, Human papillomavirus, Torque teno virus, Barmah forest virus, Chikungunya virus, Eastern equine encephalitis virus, Mayaro virus, O'nyong-nyong virus, Ross river virus, Sagiyama virus, Semliki forest virus, Sindbis virus, Venezuelan equine encephalitis virus, Western equine encephalitis virus, Junin arenavirus, Lassa virus, Lymphocytic choriomeningitis virus, Machupo virus, Pichinde virus, Human SARS coronavirus, MERS coronavirus, SARS coronavirus, Encephalomyocarditis virus, Cosavirus A, Human cytomegalovirus, Human T-lymphotropic virus, Hepatitis delta virus, Adeno-associated virus, Ebolavirus, Human rhinovirus, Coxsackievirus, Echovirus, Human enterovirus, Poliovirus, Human parvovirus B19, Murray valley encephalitis virus, Dengue virus, Japanese encephalitis virus, Langat virus, Louping ill virus, St. louis encephalitis virus, Tick-borne powassan virus, West Nile virus, Yellow fever virus, Zika virus, Hantaan virus, New York virus, Puumala virus, Seoul virus, Hendra virus, Nipah virus, Hepatitis virus, Influenza virus, Aichi virus, Human immunodeficiency virus, Cercopithecine herpesvirus, Epstein-Barr virus, Australian bat lyssavirus, Duvenhage virus, Lagos bat virus, Mokola virus, Rabies virus, European bat lyssavirus, Human astrovirus, Lake Victoria marburgvirus, Human adenovirus, Molluscum contagiosum virus, Measles virus, Human papillomavirus, Crimean-Congo hemorrhagic fever virus, Dugbe virus, Norwalk virus, Southampton virus, Oropouche virus, Bunyamwera virus, Bunyavirus La Crosse, Bunyavirus snowshoe hare, Hepatitis B virus, Human respiratory syncytial virus, Monkeypox virus, Cowpox virus, Horsepox virus, Vaccinia virus, Variola virus, Yaba monkey tumor virus, Yaba-like disease virus, Orf virus, GB virus C/Hepatitis G virus, Punta toro phlebovirus, Rift valley fever virus, Sandfly fever Naples phlebovirus (Toscana virus), Sandfly fever sicilian virus, Uukuniemi virus, BK polyomavirus, JC polyomavirus, KI Polyomavirus, Merkel cell polyomavirus, WU polyomavirus, Human parainfluenza, Rosavirus, Human herpesvirus, Rotavirus, Rubella virus, Mammalian orthorubulavirus (Simian virus), Mumps virus, Salivirus A, Sapporo virus, Banna virus, Eastern chimpanzee simian foamy virus, Simian foamy virus, Dhori virus, Vientovirus, Human torovirus, Varicella-zoster virus, Chandipura virus, Isfahan virus, and Vesicular stomatitis virus.

9. (canceled)

10. The vaccine preparation of claim 1, wherein the specific antigen(s) comprises a peptide sequence for a portion of the spike protein from SARS-CoV-2 and/or a variant thereof.

11. The vaccine preparation of claim 1, wherein the engineered MHC I peptide comprises the sequence of SEQ ID NO:2, SEQ ID NO:3, SEQ ID NO:4, SEQ ID NO:5 or SEQ ID NO:6.

12. The vaccine preparation of claim 11, wherein the engineered MHC I peptide comprises the sequence of SEQ ID NO:2.

13. (canceled)

14. The vaccine preparation of claim 1, wherein the engineered MHC II peptide comprises the sequence of SEQ ID NO:1.

15. (canceled)

16. The vaccine preparation of claim 1, wherein the vaccine preparation further comprises an adjuvant.

17. The vaccine preparation of claim 16, wherein the adjuvant is selected from aluminum hydroxide, aluminum phosphate, aluminum potassium sulfate, AS04, MF59, AS1B, and CpG 1018.

18. The vaccine preparation of claim 1, wherein the vaccine preparation is formulated for intramuscular delivery, subcutaneous delivery, intradermal, or intranasal delivery.

19. The vaccine preparation of claim 1, wherein the vaccine preparation is administered as a single dose, or as a primary dose with one or more follow up dose(s).

20. (canceled)

21. A method of making a vaccine preparation of claim 1, the method comprising:
treating an antigen presenting cell that displays engineered MHC I and MHC II peptide sequences that target specific antigen(s) or a specific epitope(s) from a pathogen with a blebbing agent comprising paraformaldehyde or N-ethylmaleimide;
isolating EBs from the antigen presenting cell;
preparing a vaccine preparation comprising the isolated EBs.

22. (canceled)

23. The method of claim 21, the method further comprising:
engineering MHC I and MHC II peptide sequences that target specific antigen(s) or a specific epitope(s) from a pathogen using a computational model of peptide vaccines for eliciting cellular immunity based upon the prediction of peptide presentation by HLA molecules from patients that were infected by the pathogen; and
introducing or expressing the engineered MHC I and MHC II peptide sequences into an antigen presenting cell.

24-33. (canceled)

34. A method for vaccinating a subject against a pathogen, comprising:
administering to the subject one or more doses of the vaccine preparation of any one of claim 1.

35. The method of claim 34, wherein the pathogen is selected from a fungus, a virus, or a bacterium.

36-44. (canceled)
